(19) United States
(12) Patent Application Publication
(10) Pub. No.: US 2026/0069119 A1
(43) Pub. Date: Mar. 12, 2026

(54) MEDICAL DEVICES, SYSTEMS, AND METHODS FOR PRESSURE SENSING

(71) Applicant: Boston Scientific Scimed, Inc., Maple Grove, MN (US)

(72) Inventors: Preston STEELE, Franklin, MA (US); Cassandra Anne HAILSON, Marlborough, MA (US); Jessica WILLIAMS, Marlborough, MA (US); Jonathan MELCHOR, Providence, RI (US); Eric WONG, South Grafton, MA (US); Niraj Prasad RAUNIYAR, Plymouth, MN (US); Vivek SHAH, Burlington, MA (US)

(73) Assignee: Boston Scientific Scimed, Inc., Maple Grove, MN (US)

(21) Appl. No.: 19/324,619

(22) Filed: Sep. 10, 2025

Related U.S. Application Data
(60) Provisional application No. 63/693,447, filed on Sep. 11, 2024.

(51) Int. Cl. A61B 1/015; A61B 1/00; A61B 5/03

(52) U.S. Cl. CPC A61B 1/015; A61B 1/00097; A61B 5/036

(57) ABSTRACT

Medical devices, systems, and methods are described, including a medical system including a medical device, a fluid management system, and a processor. The medical device may include a shaft that includes at least one working channel extending from a proximal end of the shaft to a distal end of the shaft. The fluid management system may include a first conduit in fluid communication with the at least one working channel and a pressure sensor configured to measure pressure within the first conduit. The processor may be communicatively connected to the fluid management system and configured to estimate a pressure around the distal end of the medical device based on the pressure of the first conduit measured by the pressure sensor and at least one parameter of the medical device.

### CROSS-REFERENCE TO RELATED APPLICATION(S)

This application claims the benefit of priority to U.S. Provisional Application No. 63/693,447, filed on Sep. 11, 2024, which is incorporated by reference herein in its entirety.

### TECHNICAL FIELD

Aspects of the disclosure generally relate to medical devices, systems and related methods for pressure sensing. In particular, aspects of the disclosure relate to medical systems including a processor configured to estimate an internal pressure of a bodily lumen or cavity of a patient.

### BACKGROUND

Medical devices such as scopes and sheaths are often inserted into the body of a patient to perform a therapeutic and/or diagnostic procedure inside the body. Various features of the scope and/or sheath may assist in performing a therapeutic and/or diagnostic procedure inside the subject's body, e.g., including supply of irrigation or suction. Supplying irrigation fluid into a bodily lumen or cavity of a patient during a procedure may increase the internal pressure of the bodily lumen or cavity. High internal pressures within a bodily lumen or cavity may present risks to the patient such as stressing or damaging tissue forming the bodily lumen or cavity. To decrease pressure, irrigation fluid may be removed from the bodily lumen or cavity via a suction channel. Conversely, low internal pressure within the bodily lumen or cavity may also present risks to the patient such as stressing or damaging tissue. Managing the internal pressure within the bodily lumen or cavity may divide a medical professional's attention and present additional challenges while performing a procedure.

### SUMMARY

Each of the aspects disclosed herein may include one or more features described in connection with any of the other disclosed aspects.

The present disclosure includes medical systems, devices, and related methods. For example, the present disclosure includes a medical system comprising a medical device, a fluid management system and a processor. The medical device includes a shaft extending to a distal end of the medical device. The shaft includes at least one working channel extending from a proximal end of the shaft to a distal end of the shaft. The fluid management system may include a first conduit in fluid communication with the at least one working channel and a pressure sensor configured to measure pressure within the first conduit. The processor may be communicatively connected to the fluid management system and configured to estimate a pressure around the distal end of the medical device based on the pressure of the first conduit measured by the pressure sensor and at least one parameter of the medical device.

According to some aspects of the present disclosure, the estimated pressure may be an intrarenal pressure. The at least one working channel may include a fluid channel and the first conduit may be configured to supply a fluid to the fluid channel. In some examples, the at least one working channel may include a suction channel and the first conduit may be configured to apply vacuum pressure to the suction channel. The processor may be configured to, continuously or periodically, retrieve a pressure value measured by the pressure sensor. The at least one parameter of the medical device may include a product name, a product model number, dimensions of the at least one working channel, dimensions of the shaft, or a combination thereof. The processor may be configured to retrieve the at least one parameter from an array of parameter values stored by the medical system or by a database in communication, e.g., in wireless communication, with the medical system. The processor may be configured to estimate the pressure based on a flow resistance value corresponding to the at least one parameter of the medical device.

In some examples, the at least one working channel may include a fluid channel and a suction channel. The fluid management system may further comprise a second conduit. For example, the first conduit may be configured to supply a fluid to the fluid channel and the second conduit may be configured to apply vacuum pressure to the suction channel. The pressure sensor may be a first pressure sensor and the fluid management system may include a second pressure sensor configured to measure pressure within the second conduit. The processor may be configured to estimate the pressure proximate the distal end of the medical device based on the pressure of the first conduit measured by the first pressure sensor and the pressure of the second conduit measured by the second pressure sensor.

In some examples, the processor may be configured to receive the at least one parameter from user input. For example, the medical system may further comprise a display that includes a menu of options from with a user may select the at least one parameter.

In some examples, the medical system may further comprise a sheath. The shaft of the medical device may be slidable within the sheath. A channel between an inner surface of the sheath and an outer surface of the shaft may be configured to supply fluid or apply vacuum radially outward of the shaft. The medical system may further comprise a camera or a position sensor configured to determine a position of the shaft relative to the sheath.

The present disclosure also includes a medical system comprises a medical device, a fluid management system, and a processor, wherein the medical device includes a shaft extending to a distal end of the medical device, the shaft including a first working channel and a second working channel each extending from a proximal end of the shaft to a distal end of the shaft. The fluid management system may include a first conduit and a first pressure sensor in fluid communication with the first working channel, and a second conduit and a second pressure sensor in fluid communication with the second working channel. The processor may be communicatively connected to the fluid management system and configured to estimate a pressure around the distal end of the medical device based on the pressure of the first conduit measured by the first pressure sensor, the pressure of the second conduit measured by the second pressure sensor, and at least one parameter of the medical device.

According to some aspects of the present disclosure, the at least one parameter of the medical device may include a product name, a product model number, dimensions of the first working channel, dimensions of the second working channel, or a combination thereof. The processor may be configured to receive the at least one parameter from user input. Additionally or alternatively, the processor may be configured to retrieve the at least one parameter from an array of parameter values stored by the medical system or by a database in communication, e.g., wireless communication, with the medical system.

The present disclosure also includes a medical system comprising a medical device, a fluid management system, a processor, and a display. The medical device includes a shaft that includes a first working channel and a second working channel each extending from a proximal end of the shaft to a distal end of the shaft. The fluid management system may include a first conduit and a first pressure sensor in fluid communication with the first working channel, and a second conduit and a second pressure sensor in fluid communication with the second working channel. The processor may be communicatively connected to the fluid management system and configured to estimate a pressure around the distal end of the medical device based on at least one parameter of the medical device, and at least one of the pressure of the first conduit measured by the first pressure sensor or the pressure of the second conduit measured by the second pressure sensor. The display may be communicatively connected to the processor and configured to receive the at least one parameter by user input.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 depicts an exemplary medical system including a medical device and a fluid management system.

FIG. 2 depicts a cross-sectional view of a distal portion of a shaft of the medical device of FIG. 1.

FIGS. 3A and 3B depict exemplary user interfaces of the medical system of FIG. 1.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Although scopes and ureteroscopes are referenced herein for illustration purposes, it will be appreciated that the disclosure encompasses any suitable medical device configured to allow an operator to access and view internal body anatomy of a subject and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

Aspects of this disclosure are described with reference to exemplary medical devices and systems useful for measuring pressure within the body of a subject during a medical procedure. An exemplary medical system may include a medical device, such as a scope, and a fluid management system including one or more conduits in fluid communication with the medical device. For example, the fluid management system may include a first conduit and/or a second conduit (corresponding to a fluid conduit and/or a suction conduit, respectively, or vice versa) fluidly connected to respective working channels of the medical device. The fluid conduit and/or the suction conduit may include a pressure sensor, e.g., configured to measure pressure within the fluid conduit or suction conduit. The medical system may include a processor communicatively connected to the fluid management system and configured receive parameter values from components of the medical system and/or user inputs, and/or retrieve parameter values for components of the medical system, and determine, e.g., estimate, an internal pressure of a target site. For example, the target site may be within the urinary tract or gastrointestinal tract of a subject during a medical procedure using the medical system.

Reference will now be made in detail to examples to help illustrate aspects of the present disclosure through the accompanying drawings. Wherever possible, the same reference numbers will be used throughout the drawings to refer to the same or like parts.

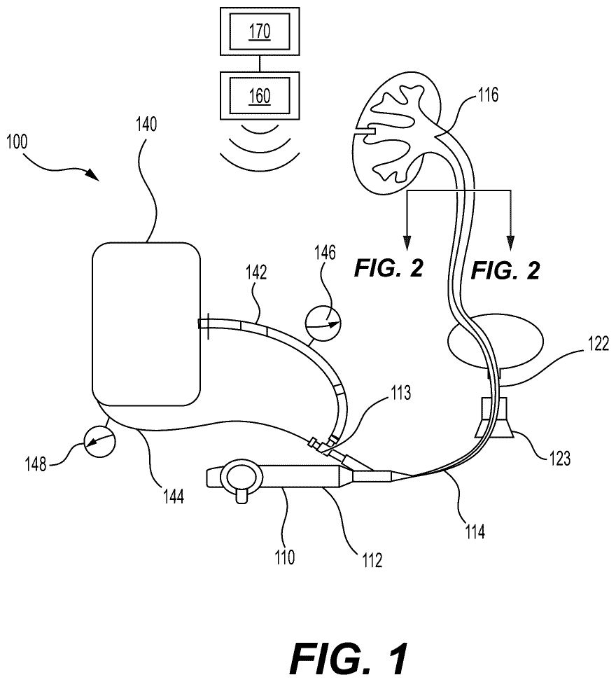

FIG. 1 depicts an exemplary medical system 100, e.g., for a medical procedure. For example, medical system 100 may be used during a lithotripsy procedure to remove a stone or calculi from a kidney or ureter of a subject. Medical system 100 comprises a medical device 110 such as a ureteroscope or other scope.

Medical device 110 may include a handle 112 and a shaft 114. Handle 112 may be configured for gripping and use by an operator. Handle 112 may include at least one port 113 in fluid communication with at least one working channel of shaft 114. Shaft 114 may be generally tubular, and may extend from a distal end of handle 112 to a distal end 116 of shaft 114. Shaft 114 includes a proximal end coupled to the distal end of handle 112. At least a portion of shaft 114 may be flexible and configured to bend or deflect during navigation toward, and away from, a target site within a body of a patient.

Shaft 114 may define one or more working channels. The one or more working channels of shaft 114 may extend from the proximal end of shaft 114 to distal end 116. A distally-facing or side facing surface at distal end 116 may include corresponding openings for each of the one or more working channels of shaft 114. The one or more working channels of shaft 114 may be configured to supply fluid or provide suction, e.g., apply vacuum, at distal end 116.

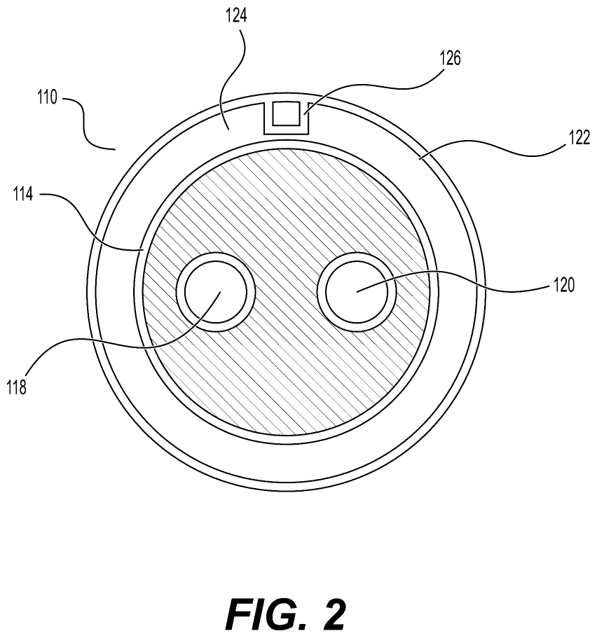

FIG. 2 depicts a cross-sectional view of shaft 114 taken at the cross-sectional cutting line shown in FIG. 1. According to some aspects, shaft 114 may include a first working channel 118 (e.g., fluid channel) and a second working channel 120 (e.g., suction channel) as illustrated in the example of FIG. 2. In other aspects, the shaft 114 may include a single working channel. Port 113 of handle 112 may be in fluid communication with first working channel 118 and/or second working channel 120. When port 113 is in communication with both working channels 118, 120, port 113 may include separate lumens or passageways to isolate the working channels 118, 120 from each other. In some examples, handle 112 includes separate ports in fluid communication with the first working channel 118 and the second working channel 120. Optionally, first working channel 118 or second working channel 120 may be sized to permit passage of a medical instrument with or without an end effector therethrough, e.g., the medical instrument introduced into medical device 110 via port 113. According to some aspects of the present disclosure, shaft 114 may include a central channel in which working channels 118, 120 are positioned therein and extend therethrough. The central channel may be configured to provide suction.

In some aspects, medical system 100 may include a sheath 122 radially surrounding and generally coaxial with shaft 114. For example, sheath 122 may be useful to provide a channel radially surrounding shaft 114 for delivery of one of fluid or suction to a target site during a medical procedure, a working channel of medical device 110 being used for delivery of the other of fluid or suction to the target site. Such capability may be desirable if shaft 114 includes only a single working channel or only one working channel available for fluid or suction (e.g., other working channels of shaft 114 being used for delivery of medical instruments or other uses). In some examples, medical system 100 does not include sheath 122.

Sheath 122 may be coupled to a handle 123 at a proximal end of sheath 122. Handle 123 may include an opening to receive shaft 114. Handle 123 may be a Y-connector, e.g., providing an opening to receive shaft 114 and a port configured to couple to a fluid source (if supplying fluid through sheath 122) or vacuum source (if providing suction through sheath 122). Shaft 114 may be slidable in the proximal or distal directions relative to handle 123 and sheath 122. Sheath 122 may include a channel 124 defined between an outer surface of shaft 114 (e.g., radially outward of shaft 114) and an inner surface of sheath 122. According to some aspects, channel 124 may be fluidly connected to a source of fluid or suction instead of second working channel 120 in the example discussed below. When medical system 100 includes sheath 122 providing channel 124 used in measuring pressure, medical system 100 also may include a position sensor or a camera 126 configured to determine a position of shaft 114 relative to sheath 122 and/or a position of sheath 122 relative to the target site (e.g., relative to a ureter of a kidney). Optionally, position sensor or camera 126 may be within channel 124. Position sensor or camera 126 may be communicatively connected to a processor (e.g., processor 160) of medical system 100 and configured to transmit, continuously or periodically, a position of shaft 114 relative to sheath 122 and/or a position of sheath 122 relative to the target site.

Referring again to FIG. 1, medical system 100 includes a fluid management system (FMS) 140. FMS 140 may be configured to provide and/or control fluid delivery and/or vacuum/suction pressure through working channel(s) 118, 120 of medical device 110 and/or channel 124 of sheath 122, e.g., at a target site. For example, for controlling vacuum/suction pressure, FMS 140 may include a vacuum pump or a peristaltic pump. According to some aspects of the present disclosure, FMS 140 may include a user interface and/or one or more actuators (e.g., buttons, knobs, joysticks, levers, etc.) for controlling fluid delivery and/or vacuum/suction pressure manually or automatically. FMS 140 may include or be communicatively connected (e.g., via wired or wireless connection) to a processor (e.g., processor 160) of medical system 100 configured to control fluid delivery and/or vacuum/suction pressure based on one or more user inputs, algorithms, and/or programs. According to some aspects of the present disclosure, another device of system 100, communicatively connected to processor 160, may control vacuum/suction pressure through working channel(s) 118, 120 and/or through conduits of FMS 140 (e.g., fluid conduit 142, suction conduit 144). For example, system 100 may include a peristaltic pump or a vacuum pump. A pressure sensor of system 100, communicatively connected to processor 160, may be configured to measure a fluid pressure within a conduit (e.g. a pressure at or proximate an end of a suction conduit 144) of the other device of system 100.

For example, FMS 140 may be configured deliver a fluid (e.g., irrigation fluid) via a working channel 118 or 120 of shaft 114 (or channel 124 of sheath 122) and control a volumetric flow rate of the fluid distally through shaft 114 (or sheath 122). FMS 140 may control one or more properties of the irrigation fluid such as, but not limited to, volumetric flow rate or temperature. The irrigation fluid may include a saline solution or sterile water. FMS 140 may include a first conduit 142 and/or a second conduit 144 configured to supply fluid and/or vacuum pressure to medical device 110 (and/or sheath 122). In the example discussed below, first conduit 142 is referred to as a fluid conduit (e.g., in fluid communication with first working channel 118 of shaft 114) and second conduit 144 is referred to as a suction conduit (e.g., in fluid communication with second working channel 120 of shaft 114) for illustration purposes. For example, first conduit 142 may be a fluid conduit fluidly connected to first working channel 118 via port 113. FMS 140 may pump fluid, or permit fluid flow, through fluid conduit 142, port 113, and first working channel 118 to the target site.

Additionally or alternatively, FMS 140 may be configured to provide and control negative pressure (suction) to medical device 110 (and/or sheath 122). For example, second conduit 144 may be a suction conduit fluidly connected to second working channel 120 via port 113. FMS 140 may provide negative pressure through suction conduit 144, port 113, and second working channel 120. Fluid within the target site may be siphoned from the target site through second working channel 120.

As shown in FIG. 1, FMS 140 may include at least one pressure sensor. While the example illustrated shows two pressure sensors, first pressure sensor 146 and second pressure sensor 148, in some aspects, the medical system 100 includes only one pressure sensor. First pressure sensor 146 may be an inlet pressure sensor connected to fluid conduit 142. For example, inlet pressure sensor 146 may be disposed along a length of fluid conduit 142, e.g., to measure pressure of fluid conduit 142. Additionally or alternatively, FMS 140 may include second pressure sensor 148 which may be an outlet pressure sensor coupled to suction conduit 144. Outlet pressure sensor 148 may be configured to measure pressure of suction conduit 144. First and second pressure sensors 146, 148 may be communicatively connected to a processor (e.g., processor 160) of medical system 100 configured to retrieve or receive pressure readings from first and second pressure sensors 146, 148. While FIG. 1 shows first and second pressure sensors 146, 148 coupled to first and second conduits 142, 144, first and second pressure sensors 146, 148 may be in other locations along the fluid or suction pathways, as long as they can measure pressure of the fluid or suction pathways. For example, first pressure sensor 146 and/or second pressure sensor 148 may be disposed along or within the corresponding working channel 118, 120 (or channel 124 of sheath 122) in order to measure pressure within the corresponding channel.

Still referring to FIG. 1, medical system 100 includes a processor 160. Processor 160 may be configured to estimate pressure at a target site, e.g., around the distal end 116 of the medical device 110 corresponding to an internal pressure of the subject at the target site. For example, as shown in FIG. 1, when distal end 116 of shaft 114 is inserted within a kidney of the patient, processor 160 may be configured to estimate an intrarenal pressure within the kidney based on measured pressure(s) of medical system 100 and at least one parameter of medical device 110. Processor 160 may be configured to receive one or more parameters of medical device 110 from one or more user inputs (e.g., via a user interface of display 170) and/or stored or in wireless communication with medical system 100. The one or more parameters of medical system 100 may include a product name, a product model number, dimensions of the working channel(s) 118, 120 (e.g., a length of first working channel 118, a length of second working channel 120, a radius or other cross-sectional dimension of the first working channel 118, a radius or other cross-sectional dimension of the second working channel 120), dimensions of the shaft 114, or a combination thereof. When the parameter(s) include a product name and/or product number, the parameter(s) may include information corresponding to components and/or dimensions of that product. Processor 160 may estimate pressure based on the parameter(s) as well as pressure measured by first pressure sensor 146 and/or second pressure sensor 148. Processor 160 may be communicatively connected to one or more components of medical system 100 and configured to receive one or more parameter values from one or more connected components. Processor 160 may estimate internal pressure at a target site (e.g., pressure around distal end 116 of medical device 110) based the parameter(s) and measured pressure(s).

Processor 160 may be communicatively connected to one or more components of medical system 100. For example, processor 160 may be communicatively connected to FMS 140. Processor 160 may be configured to control and receive parameters (parameter values) of medical system 100 such as a volumetric flow rate of fluid through fluid conduit 142. FMS 140, or another device communicatively connected to processor 160, may be configured to automatically adjust suction during a procedure to maintain a constant or approximately constant intraluminal pressure within one or more of working channels 118, 120 and/or conduits 142, 144. According to some aspects, FMS 140, or another system or device, may be configured to automatically adjust suction during a procedure to maintain intraluminal pressure within one or more of working channels 118, 120 and/or conduits 142, 144 between a predetermined lower pressure threshold and a predetermined upper pressure threshold. Processor 160 may continuously or intermittently receive or retrieve parameter values. Moreover, first and/or second pressure sensors 146, 148 may be communicatively connected to processor 160. Processor 160 may continuously or intermittently receive or retrieve pressure measurements from pressure sensors 146, 148, e.g., pressure of fluid conduit 142 or pressure of suction conduit 144. As mentioned above, other parameters of medical system 100 such as dimensions of working channels 118, 120 (e.g., length, radius, etc.) and/or the product model name or number of medical device 110 may be received or retrieved by processor 160. According to aspects of the disclosure where medical device 110 includes sheath 122, processor 160 may be communicatively connected to position sensor or camera 126 and configured to receive or retrieve a position of shaft 114 relative to sheath 122 and/or a position of the sheath 122 relative to the target site. Alternatively, a user may input one or more parameters of medical system 100, e.g., via a user interface of display 170.

Fluid dynamics relationships may be used by medical system 100, e.g., programmed into processor 160 or otherwise retrieved by processor, to estimate internal pressure at a target site of a patient based on known parameters of medical system 100 and measured pressure of fluid and/or suction provided to the target site by medical system 100. For example, processor 160 may estimate internal pressure by applying Hagen-Poiseuille's Law with the following equation (1):

$$Q = ( Δ P ) / R \qquad ( 1 )$$

where Q is volumetric flow rate through a conduit, ΔP is the change in pressure from the inlet of the conduit to the outlet of the conduit, and R is the resistance to flow of the conduit. To solve for an internal pressure at the target site, Pₜₛ, equation (1) can be rearranged into equation (1.2) or equation (1.3) as shown below, depending on which parameter values of medical system 100 are provided to or retrievable by processor 160. For example, solving for Pₜₛ,

$${P}_{ts} = {P}_{inlet} - {Q}_{in} {R}_{in} \qquad ( 2 )$$

where Pᵢₙₗₑₜ is a pressure of fluid conduit 142 (e.g., pressure at proximal end of first working channel 118), Qᵢₙ is a volumetric flow rate of fluid through fluid conduit 142 into the target site through first working channel 118, and Rᵢₙ is a resistance to flow (flow resistance value) of first working channel 118. Alternatively, solving for Pₜₛ,

$${P}_{ts} = {P}_{out} {R}_{out} + {P}_{asp} \qquad ( 1.3 )$$

where Pₐₛₚ is a pressure at or proximate an end of suction conduit 144 connected to FMS 140, Qₒᵤₜ is a volumetric flow rate of fluid out of the target site through second working channel 120, and Rₒᵤₜ is a resistance to flow of second working channel 120.

Depending on available parameters and measured pressure(s), processor 160 may estimate the internal pressure at the target site using either equation 1.2 or 1.3. In some instances, e.g., as a redundancy mechanism or for a more accurate estimate of Pₜₛ, processor 160 may calculate Pₜₛ with both equations 1.2 and 1.3 (e.g., based on pressures of both fluid conduit 142 and suction conduit 144 measured by first and second pressure sensors 146, 148).

Resistance to flow (e.g., R, Rᵢₙ, Rₒᵤₜ) is a parameter that is influenced by a number of other aspects of the medical system 100, such as dimensions of the working channel(s) 118, 120. For example, the presence or absence of a medical instrument (or other structures) within the working channel (e.g., first working channel 118), the size of the medical instrument, and position of the medical instrument may influence resistance to flow value used in estimating pressure at the target site. For example, where the working channel 118 and/or 120 does not include a medical instrument therein, resistance to flow may be approximately equal to

$$\frac{8 μ L}{π {r}^{4}} ,$$

where μ is to a viscosity of fluid flowing through the working channel, L is a length of the working channel, and r is a radius of the working channel. In other examples, where the working channel includes a medical instrument therein, more complex analytical expressions that take into account the dimensions of the medical instrument are used to calculate resistance to flow of the (e.g., through the) working channel. As will be discussed below, processor 160 may be configured to calculate or retrieve a value for flow resistance with or without a medical instrument within the working channel(s).

Processor 160, or a memory of medical system 100, may include or store one or more arrays of data for approximating or identifying a value for resistance to flow through a working channel based on retrieved (e.g., from one or more components of medical system 100) or provided (e.g., via user inputs) parameters. For example, processor 160 may identify and retrieve a value for resistance to flow of first working channel 118 from the array of parameter values after being provided with parameter(s) of a viscosity of fluid through first working channel 118, a length of first working channel 118, a radius of first working channel 118, and optionally information for a medical instrument within first working channel 118 (e.g., product name and/or model information of the medical instrument, and/or one or more dimensions of the medical instrument such as a diameter/radius), if any. As another example, processor 160 may retrieve a value for resistance to flow of second working channel 120 from the array of parameter values after being provided with a viscosity of fluid through second working channel 120, a length of second working channel 120, and a radius of second working channel 120. Processor 160 may be configured to approximate (e.g., via interpolation or extrapolation) a resistance to flow when provided parameter values are outside of a range of values of the array of parameter values or between two values of the array of parameter values. According to some aspects of this disclosure, processor 160 may be communicatively connected to a database, e.g., cloud network, and configured to transmit parameter values to the database and receive a resistance to flow value.

In some examples, via one or more user inputs, the user may provide a specific model of medical device 110 to processor 160 or processor 160 may retrieve a product name or product model number for medical device 110. Processor 160 or a memory of medical system 100 may include an array of parameter values for a plurality of medical devices including lengths and radii of working channels. After receiving the product information for medical device 110, processor 160 may retrieve parameter values specific to the selected medical device 110 and utilize associated parameter values in calculating resistance to flow of the working channel(s) 118, 120.

According to some aspects of the present disclosure, in addition to or as an alternative, processor 160 may include a calibration program configured to estimate a resistance to flow of working channel(s) 118, 120. After performing the calibration program, the processor 160 may utilize the estimated value for resistance to flow in estimating the internal pressure at the target site. The calibration program may be performed while medical device 110 and FMS 140 are in a controlled environment or when distal end 116 of shaft 114 at the target site.

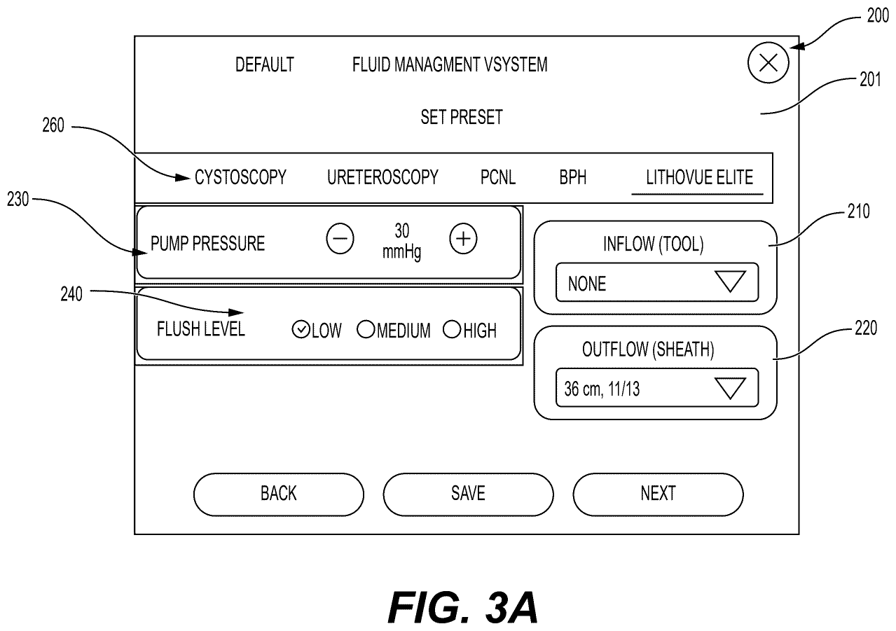

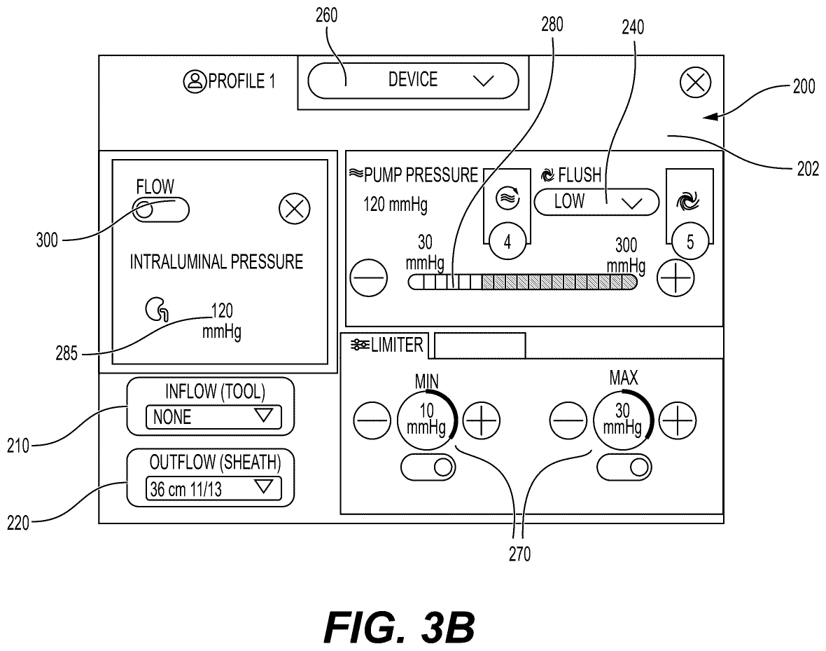

Medical system 100 may include a display 170 communicatively connected to processor 160 and a user interface 200 (FIGS. 3A-3B) displayed on display 170. User interface 200 may be configured to receive user input, e.g., via a menu of options. For example, display 170 may include a touch-screen or mouse-keyboard interface for accepting user input. A user may interact with user interface 200 to control one or more functions of FMS 140 and/or medical device 110. For example, the user may interact with user interface 200 to stop, start, increase, or decrease the volumetric flow rate of fluid through fluid conduit 142 into first working channel 118 or negative pressure through suction conduit 144 through second working channel 120.

Medical system 100 may be used during an exemplary medical procedure (optionally after calibration in a known or predetermined environment). For example, the user (e.g., a medical professional) may insert shaft 114 of medical device 110 into a bodily lumen of a subject, such as through the urethra and/or ureter, to navigate distal end 116 of shaft 114 to a target site of the subject, such as an inner cavity or lumen of a kidney. After or before navigating distal end 116 to the target site, the user may interact with user interface 200 to provide one or more parameter(s) to processor 160 and then start fluid flow and/or suction through conduits 142 and/or 144, respectively.

As discussed above, processor 160 may estimate pressure at the target site using equation 1.2 and/or equation 1.3. For example, processor 160 may estimate the internal pressure at the target site with equation 1.2 after the user provides or processor 160 retrieves the applicable product model name or number of medical device 110, a volumetric flow rate through fluid conduit 142 (e.g., Qᵢₙ, the volumetric flow into the target site), product information for a medical instrument (e.g., 200 μm Fiber, 1.9 Fr Basket, etc.) within first working channel 118 (when present), a viscosity of fluid flowing through fluid conduit 142 and first working channel 118, and the pressure of first working channel 118 measured by first pressure sensor 146. The fluid flowing through fluid conduit 142 may include a saline solution. According to other aspects of the present disclosure the fluid through fluid conduit 142 may include sterile water. Based on the user input and/or retrieved parameter values, processor 160 may retrieve or estimate the resistance to flow value of first working channel 118 from the relevant array of parameter values. After identifying the resistance to flow value of first working channel 118, processor 160 may estimate the internal pressure at the target site with equation 1.2.

In another example, processor 160 may estimate the internal pressure at the target site with equation 1.3 after the user provides, or processor 160 retrieves, a specific model of medical device 110, a volumetric flow rate through suction conduit 144 (e.g., Qₒᵤₜ, the volumetric flow rate out of the target site), a viscosity of fluid through second working channel 120, and the pressure of suction conduit 144 connected to FMS 140 from pressure sensor 148. Processor 160 may continuously or periodically re-calculate the estimated internal pressure and display the current estimated internal pressure via display 170 (e.g., shown on user interface 200; see FIGS. 3A-3B). Optionally, the viscosity of fluid through second working channel 120 may be assumed to be the same or substantially the same viscosity as the fluid through first working channel 118 discussed above. In other words, for calculation purposes, in some examples, the viscosity of fluid flowing into the target site may be assumed to be approximately the same as the viscosity of fluid flowing out of the target site via suction.

According to some aspects of the present disclosure, medical system 100 includes only one pressure sensor, e.g., first pressure sensor 146 or second pressure sensor 148. For example, processor 160 may estimate the internal pressure at the target site with equation 1.3 based on pressure of suction conduit 144 measured by pressure sensor 148 (e.g., in absence of pressure sensor 146, equation 1.2 is not used). Conversely, processor 160 may estimate the internal pressure at the target site with equation 1.2 based on pressure of fluid conduit 142 measured by pressure sensor 146 (e.g., in absence of pressure sensor 148 equation 1.3 is not used).

When medical system 100 includes medical device 110, sheath 122, channel 124, and position sensor or camera 126, processor 160 may be configured to calibrate medical system 100 based on known positions of shaft 114 relative to sheath 122 and/or sheath 122 relative to the target site. In an exemplary medical procedure, processor 160 may be configured to estimate the internal pressure of the target site with equation 1.2 or 1.3. As mentioned above, instead of second working channel 120 being fluidly connected to suction conduit 144, channel 124 may be fluidly connected to suction conduit 144, e.g., via a connection of handle 123.

Applying equation 1.3, processor 160 may estimate the internal pressure at the target site taking into account the shape and dimensions of channel 124, and the position of shaft 114 relative to sheath 122 and/or the position of sheath 122 relative to the target site. Such parameters may affect the resistance to flow value of channel 124. Accordingly, medical system 100 may include or be in communication with an array of parameter values for approximating or identifying a value for resistance to flow of channel 124 based on retrieved (e.g., from one or more components of medical system 100) or provided (e.g., via user input) parameter values. For example, processor 160 may identify and retrieve a value for resistance to flow of channel 124 from the array of parameter values after being provided with parameter values of a viscosity of fluid through channel 124 (e.g., fluid suctioned from the target site), a length of channel 124, a radius of shaft 114, a radius of sheath 122, and a position of sheath 122 relative to the shaft 114 and/or within the target site relative to the target site. In some examples, processor 160 may identify and retrieve a value for resistance to flow of channel 124 based on a position of shaft 114 relative to sheath 122 (e.g., a calibrated value for resistance to flow based on a known position of shaft 114 relative to sheath 122). In some examples, the user may provide a specific size or model of sheath 122 (e.g., 36 cm or 28 cm length, 11/13 Fr inner/outer size dimensions; etc.) to processor 160 or processor 160 may retrieve the sheath model from other components of medical system 100. In some examples, processor 160, or a memory of medical system 100, may include an array of parameter values for a plurality of sheath models including lengths and radii. After receiving the model of sheath 122, processor 160 may retrieve parameter values specific to received sheath model and utilize the respective parameter values in calculating resistance to flow of channel 124.

FIGS. 3A-3B depict exemplary user interfaces 200 of medical system 100. User interface 200 may include various menus and/or options on one or more pages, screens, or tabs. For example, user interface 200 may include a pre-procedure page 201 (FIG. 3A), and a procedure page 202 (FIG. 3B). Each of pages 201, 202 may include various input fields configured to receive user input on one or more parameters, one or more output elements configured to display information for or about medical system 100, and one or more navigation elements configured to transition between pages 201, 202 or exit user interface 200. Input fields may include drop-down menus, text boxes, sliders, check boxes, buttons, and/or other similar elements for receiving user input.

Referring to FIG. 3A, which depicts pre-procedure page 201, a user may set initial values for various parameters of medical system 100 such as volumetric flow rate into fluid conduit 142 and the presence or absence of a medical instrument through first working channel 118. For example, user interface 200 may include a tool selection field 210 that provides a list of potential medical instruments the user may select, as appropriate to the medical procedure. The list of potential medical instruments may include, but is not limited to, a 200 μm fiber, a 1.9 Fr basket, and other medical instruments appropriate for the medical procedure, or none (e.g., medical instrument absent). Tool selection field 210 may include a calibration option. Selecting the calibration option may initiate a sequence of calibration instructions in order to determine a resistance to flow through first working channel 118.

User interface 200 (e.g., page 201) may include a sheath selection field 220 providing a list of possible sheath models of which the user may select and/or a size and shape of sheath 122 (e.g., if a different sheath 122 is used). For example, the list of possible sheath models may include, but is not limited to, a 36 cm length, a 28 cm length a 11/13 Fr sheath, and other sheaths of different sizes (e.g., length, radius) appropriate for the medical procedure, or none (e.g., sheath absent). Sheath selection field 220 may include a calibration option with calibration instructions to estimate a resistance to flow through channel 124.

User interface 200 (e.g., page 201) may include a fluid field 230 for user input, e.g., to control a volumetric flow rate through fluid conduit 142. For example, fluid field 230 may control an initial pump pressure/volumetric flow rate through fluid conduit 142 and/or a pump pressure/volumetric flow rate through fluid conduit 142 during a procedure. According to some aspects, volumetric flow rate values may be displayed as pump pressure values of FMS 140. According to some aspects of the disclosure, processor 160 may be configured to convert pump pressure values to volumetric flow rate values and display the volumetric flow rate values. Processor 160 may be configured to adjust converted (e.g., from pump pressure values) volumetric flow rate values based on the sheath model selected by the user for sheath selection field 220. As shown in FIGS. 3A-3B, user interface 200 (e.g., pages 201, 202) may include a flush level field 240 for selecting a magnitude of temporary flush (e.g., volumetric flow rate of the fluid) increase provided by FMS 140 through fluid conduit 142. While flushing, a volumetric flow rate of fluid into and out of the target site may increase while intraluminal pressure through one or more working channels remains approximately the same. The FMS 140, or another aspect of system 100, may automatically increase suction provided while flushing corresponding to the increase in volumetric flow rate of fluid into the target site, e.g., to maintain intraluminal pressure of working channels 118, 120 and/or conduits 142, 144. Further, user interface 200 (e.g., page 201) may include a procedure field 260 with a menu, toggle, or tabs allowing the user to select a type of medical procedure, such as cystoscopy, a medical procedure associated with medical device 110, or other types of medical procedures.

Referring now to FIG. 3B, the user may interact with other aspects of user interface 200, e.g., via page 202, during an exemplary medical procedure. For example, during a procedure, the user may control or adjust (e.g., increase or decrease) volumetric flow rate through fluid conduit 142 and/or pressure at the target site. Page 202 may include one or more output elements (e.g., such as but not limited to, intraluminal pressure field 285) configured to display one or more of a volumetric flow rate through first working channel 118, a negative pressure through suction conduit 144, a volumetric flow rate through suction conduit 144 into FMS 140, a pressure value measured by pressure sensor 146, a pressure value measured by pressure sensor 148, and/or the pressure at the target site. Page 202 may include tool selection field 210 and/or sheath selection field 220 so that the user may select a different medical instrument within first working channel 118 or select a different sheath 122 during a procedure. Page 202 may include an intraluminal pressure field 285 configured to display pressure(s) of one or more of first working channel 118, second working channel 120, fluid conduit 142 and/or suction conduit 144.

Page 202 may include one or more limit selection fields 270, each configured so that the user may select a lower limit/threshold and/or upper limit/threshold of the pressure at the target site. Limit selection field(s) 270 may each include a toggle to return the pressure at the target site to a predetermined value and/or to a pressure value between the lower limit and the upper limit. For example, when the pressure at the target site is greater than the upper limit, FMS 140, or another aspect of system 100, may automatically increase suction to reduce pressure at the target site to a value between the lower limit/threshold and the upper limit/threshold and/or reduce pressure to a predetermined value. To adjust fluid delivery, user interface 200 (e.g., page 202) may include a pump pressure field 280 to allow the user to control, e.g., increase or decrease, volumetric flow rate through fluid conduit 142. For example, pump pressure field 280 may allow the user to increase or decrease fluid delivery via fluid conduit 142 during an exemplary procedure. Further, user interface 200 (e.g., page 202) may include one or more on/off toggles 300 for stopping and starting fluid delivery and/or negative pressure.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical system comprising:
a medical device including a shaft extending to a distal end of the medical device, the shaft including at least one working channel extending from a proximal end of the shaft to a distal end of the shaft;
a fluid management system including a first conduit in fluid communication with the at least one working channel and a pressure sensor configured to measure pressure within the first conduit, and
a processor communicatively connected to the fluid management system and configured to estimate a pressure around the distal end of the medical device based on the pressure of the first conduit measured by the pressure sensor and at least one parameter of the medical device.

2. The medical system of claim 1, wherein the estimated pressure is an intrarenal pressure.

3. The medical system of claim 1, wherein the at least one working channel is a fluid channel, and the first conduit is configured to supply a fluid to the fluid channel.

4. The medical system of claim 1, wherein the at least one working channel is a suction channel, and the first conduit is configured to apply vacuum pressure to the suction channel.

5. The medical system of claim 1, wherein the at least one working channel includes a fluid channel and a suction channel, wherein the fluid management system further comprises a second conduit, and wherein the first conduit is configured to supply a fluid to the fluid channel and the second conduit is configured to apply vacuum pressure to the suction channel.

6. The medical system of claim 5, wherein the pressure sensor is a first pressure sensor, and the fluid management system includes a second pressure sensor configured to measure pressure within the second conduit.

7. The medical system of claim 6, wherein the processor is configured to estimate the pressure proximate the distal end of the medical device based on the pressure of the first conduit measured by the first pressure sensor and the pressure of the second conduit measured by the second pressure sensor.

8. The medical system of claim 1, wherein the processor is configured to, continuously or periodically, retrieve a pressure value measured by the pressure sensor.

9. The medical system of claim 1, wherein the at least one parameter of the medical device includes a product name, a product model number, dimensions of the at least one working channel, dimensions of the shaft, or a combination thereof.

10. The medical system of claim 1, wherein the processor is configured to receive the at least one parameter from user input.

11. The medical system of claim 10, wherein the medical system comprises a display that includes a menu of options configured to receive user input selecting the at least one parameter from the menu.

12. The medical system of claim 1, wherein the processor is configured to retrieve the at least one parameter from an array of parameter values stored by the medical system or by a database in wireless communication with the medical system.

13. The medical system of claim 1, wherein the processor is configured to estimate the pressure based on a flow resistance value corresponding to the at least one parameter of the medical device.

14. The medical system of claim 1, further comprising a sheath, wherein the shaft of the medical device is slidable within the sheath, and wherein a channel between an inner surface of the sheath and an outer surface of the shaft is configured to supply fluid or apply vacuum radially outward of the shaft.

15. The medical system of claim 14, further comprising a camera or a position sensor configured to determine a position of the shaft relative to the sheath.

16. A medical system comprising:
a medical device including a shaft extending to a distal end of the medical device, the shaft including a first working channel and a second working channel each extending from a proximal end of the shaft to a distal end of the shaft;
a fluid management system including a first conduit and a first pressure sensor in fluid communication with the first working channel, and a second conduit and a second pressure sensor in fluid communication with the second working channel, and
a processor communicatively connected to the fluid management system and configured to estimate a pressure around the distal end of the medical device based on the pressure of the first conduit measured by the first pressure sensor, the pressure of the second conduit measured by the second pressure sensor, and at least one parameter of the medical device.

17. The medical system of claim 16, wherein the at least one parameter of the medical device includes a product name, a product model number, dimensions of the first working channel, dimensions of the second working channel, dimensions of the shaft, or a combination thereof.

18. The medical system of claim 17, wherein the processor is configured to receive the at least one parameter from user input.

19. The medical system of claim 18, wherein the processor is configured to retrieve the at least one parameter from an array of parameter values stored by the medical system or by a database in communication with the medical system.

20. A medical system comprising:
a medical device including a shaft extending to a distal end of the medical device, the shaft including a first working channel and a second working channel each extending from a proximal end of the shaft to a distal end of the shaft;
a fluid management system including a first conduit and a first pressure sensor in fluid communication with the first working channel, and a second conduit and a second pressure sensor in fluid communication with the second working channel;
a processor communicatively connected to the fluid management system and configured to estimate a pressure around the distal end of the medical device based on at least one parameter of the medical device, and at least one of the pressure of the first conduit measured by the first pressure sensor and the pressure of the second conduit measured by the second pressure sensor
a display communicatively connected to the processor and configured to receive the at least one parameter by user input.
